(19) United States
(12) Patent Application Publication
(10) Pub. No.: US 2004/0184962 A1
(43) Pub. Date: Sep. 23, 2004

(54) Method and device for assessing the operativeness of a device for reducing the ozone content in the air

(76) Inventors: Peter Klee, Knittlingen (DE); Dieter-Andreas Dambach, Korntal-Muenchingen (DE); Matthias Knirsch, Schwieberdingen (DE)

Correspondence Address: KENYON & KENYON, ONE BROADWAY, NEW YORK, NY, 10004, US

(21) Appl. No.: 10/473,012

(22) PCT Filed: Feb. 27, 2002
(86) PCT No.: PCT/DE02/00723

(30) Foreign Application Priority Data
Mar. 28, 2001 (DE) 101 15 219.1

(51) Int. Cl. G01N 33/00

(52) U.S. Cl. 422/83

(57) ABSTRACT

A method and a device (6, 7, 18, 20) for evaluating the operability of a DOR device (4) for reducing the ozone concentration of the air is introduced, having:

first means (6) for detecting a first ozone concentration (C1) in the air in a first action (E1) of the DOR device;

second means (7) for detecting a second ozone concentration (C2) in the air in a second action (E2) of the DOR device, the second action (E2) differing from the first (E1) action of the DOR device;

an electronic device (18) for generating a comparison result (VE) from a comparison of the first ozone concentration (C1) to the second ozone concentration (C2);

and for evaluating the operability of the system on the basis of the comparison result (VE).

### BACKGROUND INFORMATION

The present invention is directed to the diagnosis of a DOR device, i.e., a device for direct ozone reduction. What is to be reduced is the ozone concentration of the air that surrounds or flows through the device.

From U.S. Pat. No. 5,997,831, a catalytically coated radiator for a vehicle is already known via which ozone (03) in the ambient environment can be converted into oxygen (02). American emission legislation (CARS) restricts the emission of non-methane organic gases (NMOG) for each vehicle manufacturer in the form of fleet average values. In principle, it is possible to collect NMOG credits, so that the possibility exists to reduce the fleet average with respect to NMOG. In this way, the vehicle manufacturer is also able to widen the margin with respect to the lowest emission limit values (SULEV). Among others, the catalytic coating of the vehicle radiator for the purpose of reducing the ozone concentration of the ambient air likewise offers the opportunity to collect NMOG credits. The mentioned catalytic coating is referred to as direct ozone reduction (DOR) by the U.S. authorities. The designation DOR will be used in the following as well, but only as a substitute for the catalytic principle. Like any other vehicle device for reducing emissions, this system, too, must be monitored with respect to its operability within the framework of the legislated on-board diagnosis requirements.

Against this background, it is the objective of the present invention to propose methods and devices for evaluating the operability of a DOR device.

This object is achieved by the features of the independent claims.

A first specific embodiment of a device according to the present invention for evaluating the operability of a DOR device (4) for reducing the ozone concentration of the air specifically includes first means for detecting a first ozone concentration (C1) in the air in a first action of the DOR device; a second means for detecting a second ozone concentration (C2) in the air in a second action of the DOR device, this second action differing from the first action; an electronic device for generating a comparison result from a comparison of the first ozone concentration (C1) with the second ozone concentration (C2) and for evaluating the operability of the system on the basis of the comparison result.

The device according to the present invention allows the following method-step sequence in order to evaluate the operability of the DOR device:

Detecting the first ozone concentration (C1) in the air in the first action of the DOR device; detecting the second ozone concentration (C2) in the air in the second action of the DOR device, the second action differing from the first action; generating a comparison result from a comparison of first ozone concentration (C1) with second ozone concentration (C2); and evaluating the operability of the DOR device on the basis of the comparison result.

The evaluation of the operability of the DOR device according to the present invention on the basis of comparisons of the oxygen concentrations in various actions is precise, because it is based on a direct detection of the action of the DOR device on the oxygen concentration.

A preferred specific embodiment of the present invention utilizes as DOR device a heat exchanger, which is provided with catalytically acting surface coating. Since the conversion rate rises with increasing temperature of the coating, using a heat exchanger is particularly advantageous since in this case the utilizable heat is meant to be diverted anyway and is therefore available at no additional cost. Examples of heat exchangers are radiators in motor vehicles for cooling the engine, the engine oil, the oil of automatic transmissions, but also of air-condition systems, both in motor vehicles and in cooling systems in a fixed location as well as stationary cooling systems or those connected to motor vehicles.

An additional exemplary embodiment provides means to vary the action of the DOR device on the air. This allows detection of the first ozone concentration (C1) at a first instant at the same location as the detection of the second ozone concentration (C2) at a second instant, the action of the DOR device on the air being modified at the same location between the first instant and the second instant.

This has the particular advantage that a comparison measurement may be performed with only one means for detecting oxygen concentrations. In other words: In this preferred exemplary embodiment, the first means for detecting a first ozone concentration (C1) is identical to the second means for detecting a second ozone concentration (C2), so that only a single ozone sensor is required, for example.

Another specific embodiment provides that the means (12) for varying the action of DOR device (4) modifies the space velocity of the air flowing through the DOR device.

This is particularly advantageous due to the fact that the conversion rate at which the DOR device converts ozone has a marked dependency on the space velocity. In a vehicle in which the wind from driving flows through the DOR device, the space velocity is in turn dependent on the vehicle velocity and the radiator-fan speed, for example. Both variables may be influenced in a simple and reproducible manner. Alternatively, variations in the driving speed that occur naturally during driving are able to be used in a reproducible manner to detect different actions of the DOR system by conversion rates having different rates.

As an alternative, it is possible in another exemplary embodiment to detect the first ozone concentration C1 at a first location, spatially separate from the detection of the second ozone concentration C2 at a second location. For example, two ozone sensors as first and second means for detecting the ozone concentration may be spatially arranged in such a way that the action of the DOR device on the air changes between the first and the second location. In the case of a vehicle radiator, this corresponds to disposing one sensor upstream and one sensor downstream from the radiator, which has the advantage that the first and the second concentrations may be ascertained simultaneously or in rapid succession.

A further specific embodiment provides that the first means for detecting a first ozone concentration C1 at a first location is moved to a second location for detecting the second ozone concentration C2. For instance, an ozone sensor is moved into the air stream behind the DOR device and then moved out again.

This has the advantage that one sensor is able to detect two different actions of the DOR device without modification of the air flow.

Additional advantageous embodiments may be gathered from the following description, which makes reference to the figures.

The figures show:

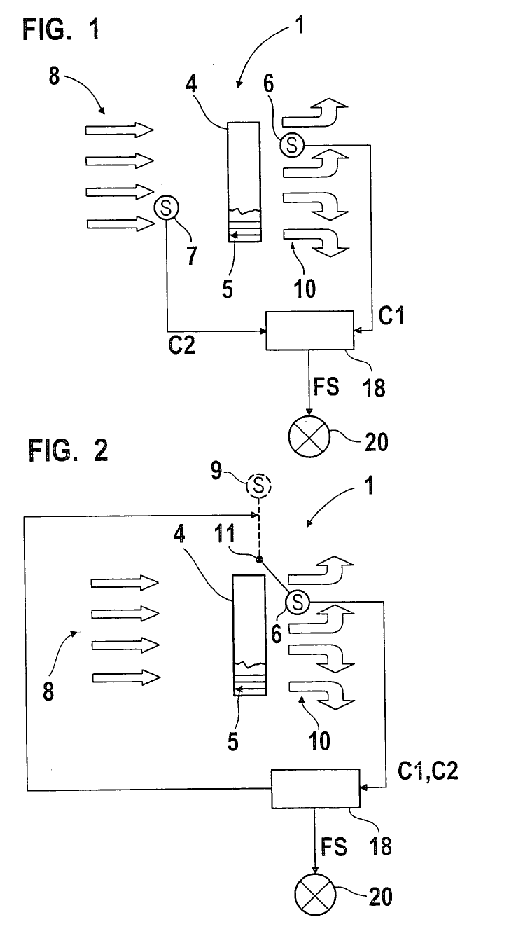

FIG. 1 a first exemplary embodiment of a device according to the present invention, which has two means 6 and 7, which are spatially separate and are used to detect the ozone concentration.

FIG. 2 a first specific embodiment of the present invention with only one means 6 for detecting an ozone concentration.

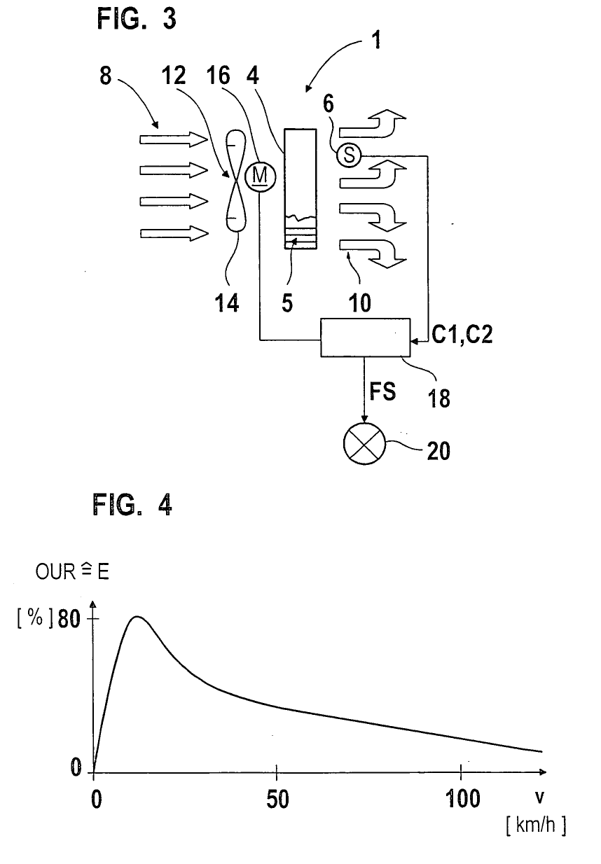

FIG. 3 another specific embodiment of the present invention with only one means 6 for detecting an ozone concentration.

FIG. 4 the profile of the ozone conversion rate OUR, which corresponds to a measure of action E of the DOR device on the air, as a function of the vehicle speed or the space velocity of the air through the DOR device.

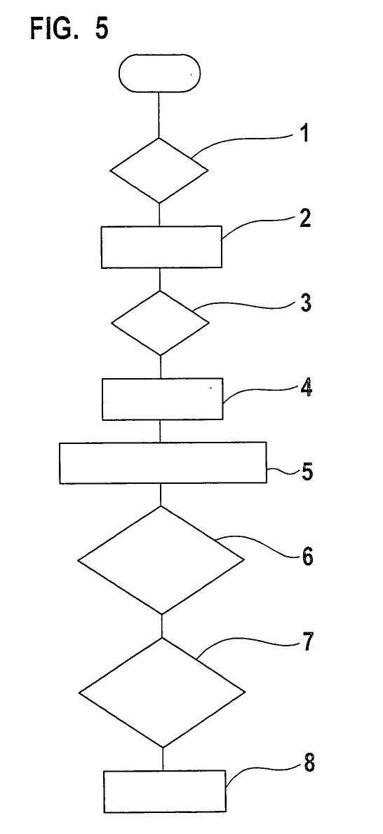

FIG. 5 a flow chart as exemplary specific embodiment of a method according to the present invention.

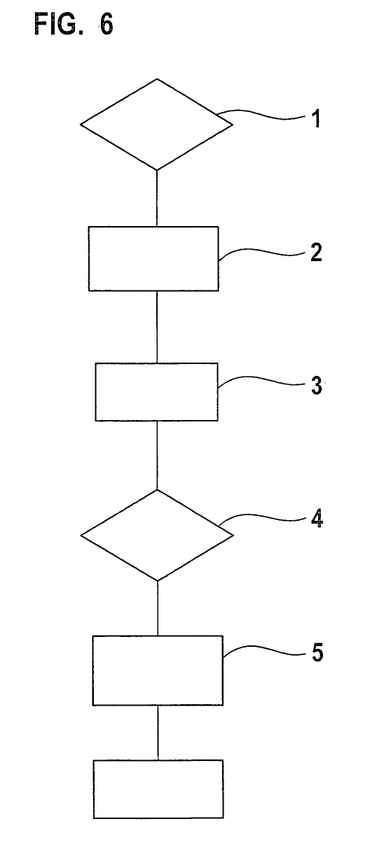

The flow chart of FIG. 6 is able to be executed in particular in connection with the devices according to FIG. 2 and FIG. 3.

### Specification

Numeral 1 in FIG. 1 denotes the spatial arrangement of a DOR device 4 and additional components within the framework of an exemplary specific embodiment of the present invention. The DOR device has catalytically coated surfaces 5. Numeral 6 denotes a first means for detecting an ozone concentration, such as an ozone sensor. It detects the ozone concentration C1 of the air subsequent to the action of the DOR device. This air is represented by the arrows denoted by numeral 10. Numeral 7 correspondingly denotes a second means for detecting the ozone concentration of the air. This means detects ozone concentration C2 of the air in a second action of the DOR device, the second action differing from the first action. In the example of FIG. 1, this air is represented by the arrows denoted by numeral 8 in front of the DOR device, and the second action is equal to 0. Correspondingly, the arrows denoted by numeral 10 denote the air in a second action of the DOR device. In the example of FIG. 1, this air corresponds to the air subsequent to the action of DOR device 4.

The signals of both means 6 and 7, which stand for the first (C1) and the second (C2) ozone concentration, are forwarded to an electrical device 18 for conditioning and evaluation. The evaluation includes the generation of a comparison result VE from a comparison of first ozone concentration C1 to second ozone concentration C2 and an evaluation of the operability of the DOR device on the basis of the comparison result. If a comparison results in an insufficient operability, a corresponding error report is output or stored. The output may occur, for example following a statistical safeguard, by a warning light 20 in the visual field of the driver of a motor vehicle equipped with a DOR device.

FIG. 2 shows a first specific embodiment of the present invention with only one means 6 for detecting an ozone concentration. Numeral 9 in FIG. 2 denotes means 6 in its second position (dashed line). In the position indicated by a solid line, means 6 detects the ozone concentration subsequent to the action of the DOR device, i.e., in a first action E1 of the DOR device. In position 9, denoted by a dashed line, means 6 detects the ozone concentration outside of the air flowing out of the DOR device, and thus in a second (E2) action of the DOR device, which differs from the first (E1) action E of the DOR device. The positional change may be implemented electromechanically, for example, and be triggered by electronic device 18. In this manner, the detection of first ozone concentration C1 is carried out at a first location, spatially separate from the detection of second ozone concentration C2 at a second location, with the aid of a position change of a single means 6. In a catalytically coated vehicle radiator, the ozone sensor may be moved with the aid of a suitable device, for example, so that it may measure, on the one hand, the raw ambient air before it flows through the vehicle radiator and, on the other hand, the ambient air after it has flowed through the vehicle radiator.

FIG. 3 shows another specific embodiment of the present invention with only one means 6 for detecting an ozone concentration. In this case, the ozone concentration is detected in different actions of the DOR device, not with spatial but with temporal separation. First ozone concentration (C1) is detected at a first instant T1 at the same location in which second ozone concentration (C2) is detected at a second instant T2.

In an operative DOR device, the ozone concentrations at both instants will differ when action E of the DOR device on the air was modified or has changed between both instants.

Suitable for modifying action E, for example, is a change in the space velocity at which the air flows through the DOR device. Here, space velocity is understood to mean the air volume flowing through the DOR device per time unit. In the case of a motor vehicle, for example, the air volume corresponds to a vehicle velocity v. In a vehicle radiator, given a fixed position of an ozone sensor, for instance, the ozone may be measured in different vehicle and engine operating conditions. As a function of different vehicle and engine operating conditions, there is either raw ambient air at the location of the ozone sensor, or ambient air that has flowed through the vehicle radiator.

Alternatively, in a fixed position of the sensor, it is possible to prevent the flow through the radiator temporarily. By temporarily preventing the flow through of the vehicle radiator, for example via a radiator shutter or by a switchable supply of ambient air (radiator bypass), it is possible to ensure that raw ambient air is present at the ozone sensor at one instant, and converted air at another instant.

A simple specific embodiment uses a comparison measurement between raw ambient air and ambient air subsequent to flowing through the catalytically coated vehicle radiator. To diagnose the conversion capability of a DOR system with the aid of one ozone sensor, the ozone sensor must be secured in the vicinity of the vehicle radiator in such a way that the air flowing through the vehicle radiator may be measured.

For all three measuring principles, the type and manner of the airflow through the vehicle radiator in different vehicle and engine operating points is advantageously measured within the framework of a basic test and the results are stored in characteristics maps of the vehicle control device, so that reference value suitable for the ozone measurement are able to be accessed during serial operation of the vehicle. This relates in particular to the measurement of the air after it has flowed through the radiator, which depends on various conditions, such as engine-coolant temperature, engine speed, cooling fan speed, vehicle speed etc.

Among others, the following effects, in particular, may be used for the comparison measurement:

decreasing ozone conversion at increasing space velocity;

no ozone conversion without through-flow (space velocity=zero).

Examples for realizing a diagnosis function with a device according to FIG. 3:

A) Evaluating the difference of the 03-concentration in a stationary and a moving vehicle: Fixed placement of the 03-sensor behind the radiator, that is, between radiator and engine. First measurement of the ozone concentration of the air surrounding the sensor as soon as the following marginal conditions have been met for a predefined duration (such as 30 seconds):

engine has operating temperature (radiator exhibits good 03 conversion rate);

vehicle is stationary (v=0);

engine fan wheels are at standstill.

The second measurement occurs at the next standing/start, as soon as the vehicle speed has reached a predefined value (such as 8 km/h).

If the DOR device works correctly, the difference between the two measured 03 concentration values must exceed a certain predefined value.

B) Evaluating the difference in the 03 concentrations at at least two different vehicle speeds: Fixed placement of the 03-sensor behind the radiator, that is, between radiator and engine. First measurement of the ozone concentration of the air surrounding the sensor as soon as the following marginal conditions have been satisfied:

the engine has operating temperature;

the vehicle drives at low speed within a speed range to be predefined (such as v=6 . . . 8 km/h);

the engine fan wheels are at standstill.

The second measurement is taken if the vehicle speed, within a predefined time range following the first measurement (30 seconds, for example), attains a predefined value (such as 70 km/h).

If the DOR device operates correctly, the difference between the two measured 03 concentration values must exceed a certain predefined value, since the 03 conversion rate decreases above approximately 5 km/h with increasing speed. If both measured values are close to one another, this is an indication that the DOR device no longer works properly.

C) Evaluating the difference in the 03 concentration when the vehicle is stationary before and after switching on the fan:

Fixed securement of the 03-sensor in the flow direction behind the radiator fan, between radiator and combustion engine.

The first measurement of the ozone concentration of the air surrounding the sensor occurs as soon as the following marginal conditions have been met for a duration to be predefined (such as 30 seconds):

engine has operating temperature (radiator exhibits good 03 conversion rate, for instance at Tmot>80° C.);

vehicle is stationary (v=0);

engine fan wheels are at standstill.

Then the fan is switched on for diagnostic purposes and a second measurement of the ozone concentration is conducted. The fan generates a flow through the radiator, a functioning DOR device reducing the ozone concentration of the air conveyed to the 03 sensor by the fan.

If the DOR device is working properly, the difference in the values of the two measured 03 concentrations must exceed a certain predefined value.

To increase the diagnosis reliability, a statistical evaluation of a plurality of comparison measurements may be implemented, which also results in a final diagnosis result. This evaluation may extend over several trips.

FIG. 4 shows the profile of ozone conversion rate OUR, which corresponds to a measure of action E the DOR device on the air, as a function of the vehicle speed or the space velocity of the air through the DOR device. The ozone conversion rate able to be obtained in practice depends to a slight degree on the temperature of the coating or on the absolute ozone concentration, to a large degree on the space velocity of the ambient air through the vehicle radiator.

In the specific embodiment shown in FIG. 3, the ozone concentrations C1, C2 are detected, for example, at different space velocities and thus in different actions. In this sense, the vehicle drive constitutes a means 12 for modifying the action, so to speak. In addition or as an alternative to modifying the space velocity by a change in the vehicle velocity, the space velocity, and thus the action, may also be realized by a change in the air supply of a ventilator 14. For example, the rotational speed of an electromotor 16 driving the ventilator may be appropriately adjusted by electronic device 18.

FIG. 5 shows a flow chart as exemplary specific embodiment of a method according to the present invention. After starting the method, it is checked in step 1 whether action E of the DOR system corresponds to a first action E1, which is suited to detect first concentration C1. For example, the sensors must be operative and the catalytic surfaces should have operating temperature. In step 2, ozone concentration C1 is then measured in first action E1. If the conditions for detecting second ozone concentration C2 are satisfied (step 3), oxygen concentration C2 is detected in step 4 in second action E2. Subsequently, a comparison result VE is formed in step 5 as a function of measured ozone concentrations C1, C2 and possibly additional parameters, such as the temperature at the testing instant etc. In steps 6 and 7, the comparison result is compared to predefined limits, which define a good range (step 6) and a poor range (step 7). If the comparison result corresponds to an element from the poor range, an error signal is emitted in step 8. The comparison result is in the good range, for example, when the difference in the two measured ozone concentrations exceeds a certain predefined value. The specific embodiment according to FIG. 5 may be used in connection with the devices according to FIG. 1, 2 or 3. In connection with FIG. 2, the query in step 1 of FIG. 5 includes, for example, the check whether the means for detecting the ozone concentration is in position 6 or in position 9. In this context, condition E=E1 corresponds to position 6. Analogously, condition E=E2 corresponds to position 9. In connection with FIG. 3, condition E=E1 corresponds to a driven fan 14, and condition E=E2 corresponds to a fan wheel at standstill. Analogously, E=E1 may correspond to a moving vehicle and E=E2 to a stationary vehicle or a vehicle driven at lower speed.

The flow chart of FIG. 6 is executable in particular in connection with the devices according to FIG. 2 and FIG. 3. Again it is checked in a step 1 whether action E of the DOR system corresponds to an action E1, which is suitable for detecting an ozone concentration C1. If this is the case, ozone concentration C1 is detected in step 2 at instant T=T1. Subsequently, action E is varied in step 3. Possibilities for varying action E on the air at the sensor location are: changing the sensor positions as shown in FIG. 2; changing the space velocity of the air through the DOR device by modifying a vehicle velocity or by changing a fan speed. Subsequently, it is checked in step 4 whether action E corresponds to an action E2, which is suitable for detecting an ozone concentration C2. For this purpose, it may be checked, for instance, whether the vehicle velocity is within a predetermined range or whether the fan is driven at a certain output. If this is the case, ozone concentration C2 is detected in step 5 at instant T=T2. In the further course, the method is continued by the step according to FIG. 1.

What is claimed is:

1. A device (6, 7, 18, 20) for evaluating the operability of a DOR device (4) for reducing the ozone concentration of air, having
first means (6) for detecting a first ozone concentration (C1) in the air in a first action (E1) of the DOR device;
second means (7) for detecting a second ozone concentration (C2) in the air in a second action (E2) of the DOR device, the second action (E2) differing from the first (E1)of the DOR device;
an electronic device (18) for generating a comparison result (VE) from a comparison of the first ozone concentration (C1) to the second ozone concentration (C2);
and for evaluating the operability of the system on the basis of the comparison result (VE).

2. The device as recited in claim 1 having as DOR device a heat exchanger, which is provided with catalytically acting surface coatings (5).

3. The device as recited in claim 1 or 2, having means (12) for varying the action of the DOR device (4) on the air.

4. The device as recited in claim 3, wherein the means (12) for varying the action of DOR device (4) modify the space velocity of the air flowing through the DOR device.

5. A method for evaluating the operability of a DOR device (4) for reducing the ozone concentration of air, having the steps:
detecting a first ozone concentration C1 in the air in a first action E1 of the DOR device (4);
detecting a second ozone concentration C2 in the air in a second action (E2) of the DOR device (4), the second action (E2) differing from the first (E1) action of the DOR device (4);
generating a comparison result VE from a comparison of the first ozone concentration C1 to the second ozone concentration C2;
evaluating the operability of the DOR device (4) on the basis of the comparison result.

6. The method as recited in claim 5,
wherein the detection of first ozone concentration C1 is implemented at a first location, spatially separate from the detection of second ozone concentration C2 at a second location.

7. The method as recited in claim 6,
wherein the action of the DOR device (4) on the air changes between the first and the second location.

8. The method as recited in claim 6,
wherein the first means (6) for detecting a first ozone concentration (C1) at a first location is moved for detecting the second ozone concentration C2 at a second location.

9. The method as recited in claim 1,
wherein the detection of the first ozone concentration (C1) takes place at a first instant at the same location as the detection of the second ozone concentration (C2) at a second instant.

10. The method as recited in claim 5, wherein the action of the DOR device (4) on the air is varied at the same location between the first instant and the second instant.
